(19) United States
(12) Patent Application Publication
(10) Pub. No.: US 2010/0256575 A1
(43) Pub. Date: Oct. 7, 2010

(54) ADAPTER FOR CANNULAS FOR DISPENSING PRODUCTS AND CANNULA THEREFOR

(76) Inventors: Alessio Nobbio, Gargagnate Milanese (MI) (IT); Dolores Blanco Lousame, Alcobendas (Madrid) (ES); Jaime Moscoso Del Prado, Alcobendas (Madrid) (ES)

Correspondence Address: HEDMAN & COSTIGAN, P.C., 1230 AVENUE OF THE AMERICAS, 7th floor, NEW YORK, NY, 10020, US

(21) Appl. No.: 12/734,650

(22) PCT Filed: Nov. 24, 2008
(86) PCT No.: PCT/IB2008/003298

§ 371 Date: Jun. 10, 2010

(30) Foreign Application Priority Data
Nov. 29, 2007 (IT) MI2007A002251

(51) Int. Cl. A61M 25/00

(52) U.S. Cl. 604/264

(57) ABSTRACT

Adapter (3) for cannulas (2) for dispensing products comprising a first hollow substantially cylindrical portion (31) provided inside with means for fastening to a bottle (4) containing such products to be dispensed with the cannula and a second cylindrical portion (32) that joins to the first portion having a channel (33) that connects to an opening located on the mouth (41) of the bottle (4) and that is suitable for receiving the end portion of the cannula.

The present invention refers to an adapter for cannulas for dispensing medicinal products, in particular of the type used to apply semisolid vaginal and/or anal or haemorrhoidal products, and the cannula to be used in association with such an adapter

Currently, packs of medicinal products for vaginal, anal or haemorrhoidal use normally comprise a container of the medicinal product and a series of cannulas, usually coming in sixes, eights or tens able to be used for application.

Conventional cannulas, indeed, comprise a collection tube or cylinder, inside of which a piston slides that is fixedly connected to an actuation shaft.

The tube usually has a threaded end to allow the container of the medicinal product to be associated there, by screwing it in. The opposite end is perforated to allow the actuation shaft to partially come out from the tube, so that the cannula can be used correctly, firstly by pulling back the shaft and taking in the product inside the tube and then letting out the medicinal product at the threaded end, under the thrust of the flat surface of the piston, when it is made to slide in the opposite direction to that for taking in the product, through the actuation shaft. Regarding this, the piston is optionally shaped and has a larger diameter than the diameter of the actuation shaft and substantially equal to the internal diameter of the tube, so as to slide with a certain friction along the inner walls of the tube, to ensure the seal of the coupling during the axial movement of the shaft, during application of the product, and to ensure that most of the product sucked in earlier is pushed towards the outlet, without it escaping beyond the collection surfaces of the piston.

These known cannulas do, however, have numerous drawbacks, like for example the need to have a minimum diameter at least greater than the outer diameter of the end of the container or bottle or tube containing the product to allow the association thereof. Moreover, in the case in which such an association takes place by screwing, the end of the cannula must necessarily have a threading and therefore a profile with sharp edges, which represent a drawback for objects that must be introduced in delicate positions, for example anal or vaginal cavities.

Moreover, for hygienic reasons, it is absolutely necessary for single-use tubes and shafts to be made and this results in a substantial consumption of material, a substantial space for storage and transportation, and a substantial increase in costs, according to the number of applications foreseen.

The purpose of the present invention is to avoid the aforementioned drawbacks, by making an adapter that is arranged between the cannula and the bottle that contains the product to be dispensed, which allows the outer diameter of the collection tube to be reduced to the minimum and allows an “ogive-shaped” profile, limiting the invasive action of the cannula for dispensing vaginal, anal or haemorrhoidal products as much as possible, at the same time ensuring extremely low overall production, storage and transportation costs of the cannula compared to the prior art.

This and other purposes, according to the present invention, are accomplished by an adapter for cannulas for dispensing semi-solid medicinal products, according to claim 1, and the cannula to be used with such an adapter according to claim 8 to which we refer for the sake of brevity.

Advantageously, according to the invention, it is possible to limit as much as possible the consumption of throwaway material at each application of product, consequently reducing the overall production, storage and transportation costs and also reduce the invasive action of the cannula in the vaginal or anal cavity resulting in greater comfort for the patient and ensuring the effectiveness of the medical treatment.

Further characteristics and advantages of the adapter according to the present invention shall become clearer from the following description, given as a non-limiting example referring to the attached schematic drawings, in which:

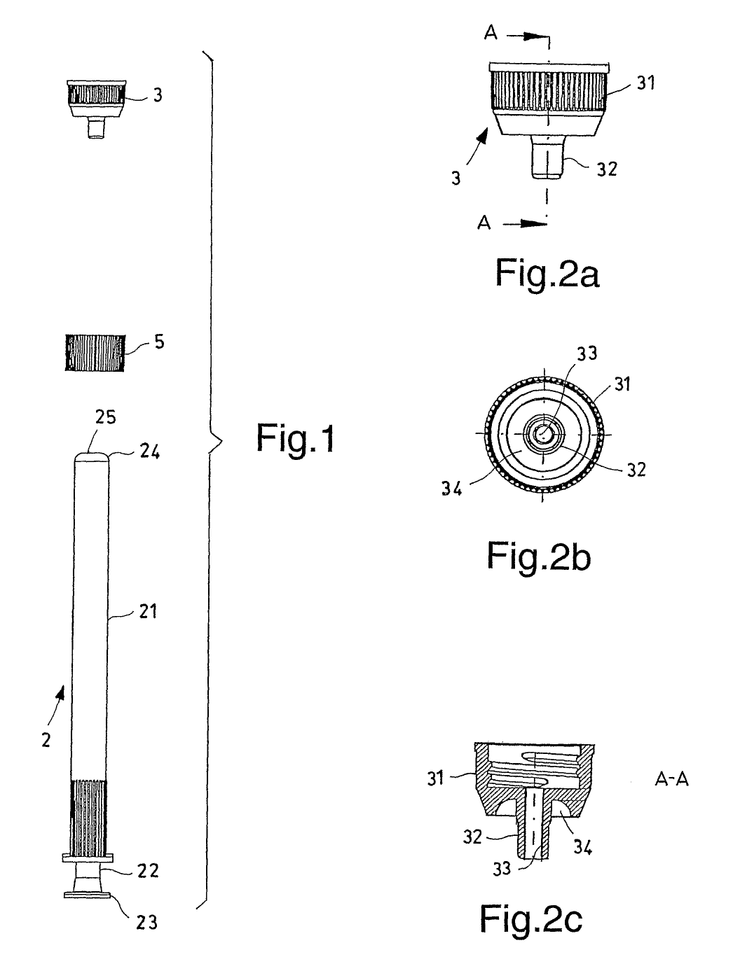

FIG. 1 is an exploded schematic view of a cannula and of an adapter according to an embodiment of the invention;

FIGS. 2a, 2b and 2c illustrate the adapter respectively in a side view from above and in section according to a first embodiment of the invention;

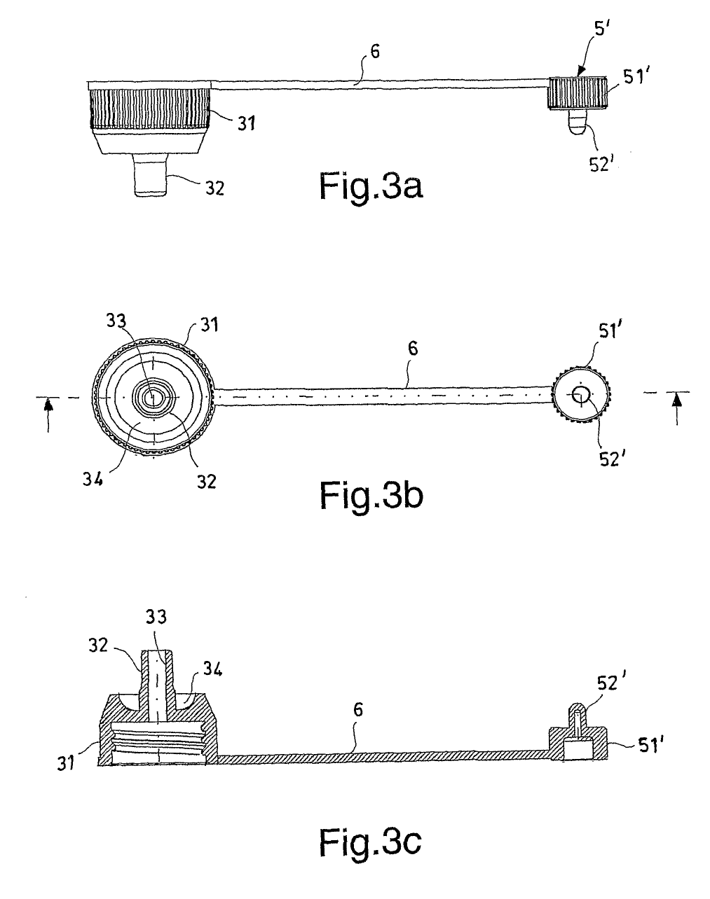

FIGS. 3a, 3b and 3c illustrate the adapter respectively in a side view from above and in section according to a second embodiment of the invention;

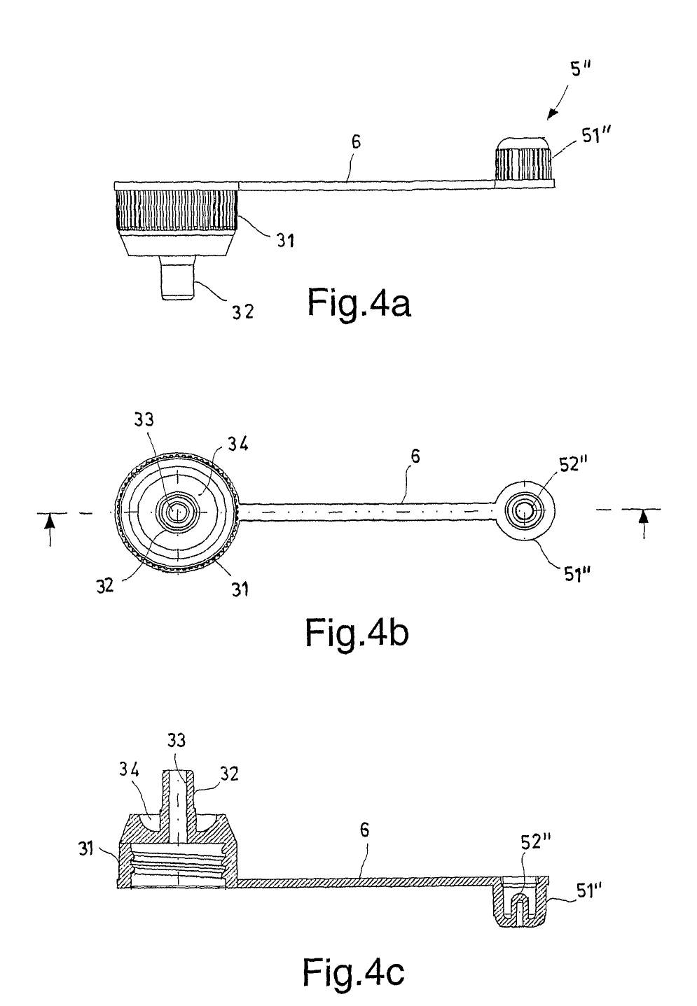

FIGS. 4a, 4b and 4c illustrate the adapter respectively in a side view from above and in section according to a third embodiment of the invention;

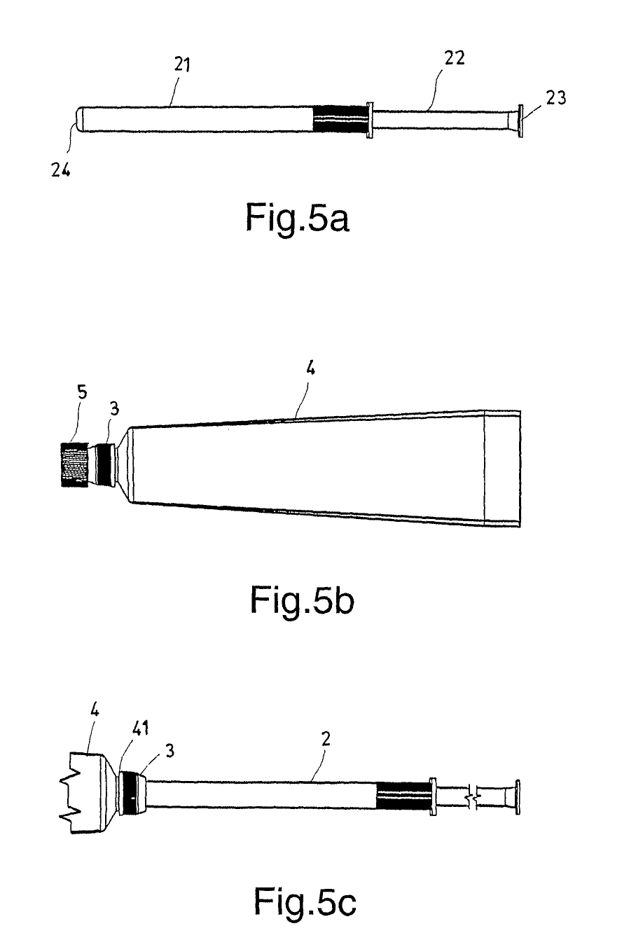

FIG. 5a illustrates the cannula with the piston inserted inside it according to the present invention;

FIG. 5b illustrates the bottle with the adapter screwed and the cap fixed onto it according to the present invention;

FIG. 5c illustrates the adapter without the cap and with the cannula inserted into it according to the present invention.

With reference to the quoted figures, a cannula 2 for dispensing medicinal products, for example vaginal, anal or haemorrhoidal ointments, salves or gels, according to the present invention is illustrated that comprises a tube or cylinder 21 inside of which a small piston 22 can slide, which can be actuated by a user from the outside through a disc-shaped end portion 23. At one end of the tube an ogive-shaped portion 24 is formed and it has a hole 25 to allow the passage of the product and at the opposite end of the tube the quoted piston is inserted.

In an intermediate area of the cylinder 21 an undercut is made, which acts as a stop against the stroke of the piston, which is preferably made through a raised edge, inside the cylinder, which extends along at least an arc of circumference thereof and engages on an upper ring of the piston, which takes care of ensuring the seal of the piston-cylinder coupling during the movement of the piston inside the cannula.

Preferably, the side surface of the end of the cylinder 21 closest to the disc 23 is rough or knurled to make the cannula easier to grip.

The adapter 3 according to the present invention comprises a first hollow substantially cylindrical portion 31 provided inside with means for fixing a mouth 41 of a bottle 4 containing the products to be dispensed with the cannula. Such fixing means in the illustrated embodiments preferably consist of a threading.

A second cylindrical portion 32 joins to the first and has a channel 33 that connects to an opening 42 normally located on the mouth 41 of the bottle 4 and that is suitable for receiving the end portion of the cannula. Advantageously, the first cylindrical portion has the side surface rough or knurled so as to make the adapter easier to grip.

The adapter is preferably provided with a cap 5, which is engaged on such a second portion making sure that the bottle is kept closed once the adapter is screwed or fixed onto the mouth of the bottle itself, after it has previously been opened or had its original lid removed.

In the joining area between such a first portion and the second portion an annular notch 34 is foreseen suitable for making it easier for such a cap to engage in the adapter itself.

In the embodiments illustrated in FIGS. 3 and 4 the adapter and the cap are joined together by means of a flexible strip 6. In particular, in FIGS. 3a, 3b and 3c the cap 5′ comprises a projection 52′ and a cylindrical base element 51′. The projection inserts into the channel 33 of the adapter so as to plug the bottle once the adapter itself is in operative position fixedly connected to the bottle. Preferably, the side surface of the cylindrical base element 51′ is rough or knurled to make it easier to grip the cap.

In FIGS. 4a, 4b and 4c the cap 5″ comprises a hollow cylindrical base element 51″ inside of which a projection 52″ is formed. The projection inserts into the channel 33 of the adapter so as to plug the bottle once the adapter itself is in operative position fixedly connected to the bottle when the cylindrical base portion covers the second cylindrical portion 32. Preferably, the side surface of the cylindrical base element 51′ is rough or knurled to make it easier to grip the cap.

The operation of the adapter and of the cannula according to the present invention is the following.

As shown in FIGS. 5a, 5b and 5c the adapter 3 is firstly mounted onto the threaded mouth 41 of the bottle 4 and then the cannula 2 is mounted by inserting the piston into the hole of the cylinder 21.

Then, the end portion of the cannula that has the ogive 24 and the hole 25 is inserted into the channel 33 of the adapter and, then, the medicinal product can be introduced inside the tube of the cannula by pressing the bottle.

In this step, the flow of the product coming out from the bottle progressively pushes the piston, initially associated in abutment with the undercut of the end. Then, to proceed to apply the medicinal product it is sufficient to actuate the piston.

The medicinal product preferably has semi-solid consistency, for example like a ointment, salve, gel, emulsions or creams, preferably to be administered vaginally, anally or haemorrhoidally.

Finally, it is clear that numerous variants can be brought to the adapter and to the cannula according to the present invention, without for this reason departing from the novelty principles inherent to the inventive idea, just as it is clear that, in the practical embodiment of the invention, the materials, the shapes and the sizes of the illustrated details can be whatever according to requirements and they can be replaced with others that are technically equivalent.

1. Adapter (3) for cannulas (2) for dispensing products characterised in that it comprises
a first hollow substantially cylindrical portion (31) provided on its inside with means for fastening to a bottle (4) containing such products to be dispensed with the cannula,
a second cylindrical portion (32) that joins to the first portion having a channel (33) that connects to an opening located on the mouth (41) of the bottle (4) and that is suitable for receiving the end portion of the cannula.

2. Adapter according to claim 1, wherein such fastening means consist of a threading.

3. Adapter according to claim 1, also comprising a cap (5), which is engaged on such a second portion (32) taking care to keep the bottle closed once the adapter is screwed or fixed onto the mouth of the bottle itself, after it has previously been opened or had its original lid removed.

4. Adapter according to claim 3, wherein in the joining area between such a first portion and the second portion an annular notch (34) is foreseen suitable for promoting the engagement of such a cap in the adapter itself.

5. Adapter according to claim 1, wherein the adapter itself and the cap are joined together by means of a flexible strip (6).

6. Adapter according to claim 3, wherein such a cap (5′) comprises a projection (52′) and a cylindrical base element (51′), such a projection being suitable for inserting into the channel (33) of the adapter so as to plug the bottle.

7. Adapter according to claim 3, wherein such a cap (5″) comprises a hollow cylindrical base element (51″) inside of which a projection (52″) is formed, such a projection being suitable for inserting into the channel (33) of the adapter so as to plug the bottle.

8. Cannula suitable for being used with the adapter according to claim 1, characterised in that it comprises a substantially cylindrical tube (21) having an ogive-shaped end (24) and having a hole (25) that associates with the channel (33) of the adapter to allow the passage of the product.

9. Cannula according to claim 8, wherein inside such a tube or cylinder (21) a small piston (22) can slide, able to be actuated by a user from the outside through a disc-shaped end portion (23).
